(19) United States
(12) Patent Application Publication
(10) Pub. No.: US 2012/0209154 A1
(43) Pub. Date: Aug. 16, 2012

(54) HOT AND COLD THERAPY DEVICE

(76) Inventors: Riley J. Williams, III, New York, NY (US); Roy Swan, JR., New York, NY (US)

(21) Appl. No.: 13/025,077

(22) Filed: Feb. 10, 2011

(51) Int. Cl. A61H 1/00; A61F 7/00

(52) U.S. Cl. 601/19

(57) ABSTRACT

A device and method for applying compressive hot or cold therapy to a patient's body, including a handle and a frame connected to and extending from the handle. The frame includes two spaced apart support arms, the support arms each having an opening such that the openings in the support arms are collinear with one another. A roller having a central axis and a reservoir within its interior for retaining a thermal medium, the roller having a non-uniform cross-section along its length defining a thermally conductive external surface for transferring thermal energy from the thermal medium to the patient's body. At least one elongated member is operatively connected to the roller and extends longitudinally along the central axis beyond the periphery of the roller. The elongated member is received within the collinear openings of the spaced apart support arms to releasably and rotatably connect the roller to the frame.

### BACKGROUND

1. Field

This disclosure relates generally to devices for treating a desired region of a patient's body, and, more particularly, to a thermal therapy massage roller for application of heat or cold to an affected area on a patient's body.

2. Background

Common injuries, such as (but not limited to) joint sprains, muscle strains, shin splints, and bursitis of the knee, elbow, shoulder, leg, hip, trunk or ankle, often propagate an inflammatory response. Treatment of such injuries and other painful, inflammatory disorders of the muscles, ligaments, tendons, bones and joints may include the application of localized heat and/or cold temperature to the afflicted area of the patient's body. For instance, cryotherapy is an effective modality for the reduction of pain caused by inflammation. Cold material may be placed against the affected area of a patient's body, thereby absorbing heat from the body through thermal conduction. This may trigger a variety of beneficial physiological effects. For example, the application of cold temperature to the afflicted area may produce a numbing effect, reducing the pain experienced by the injured patient. It may also trigger vasoconstriction, which, by restricting the blood flow to the affected area, may reduce swelling. Other beneficial effects may be produced, such as a reduction in local metabolic rate, or relief from uncontrolled muscle spasms. In addition, cold therapy is a known anti-inflammatory modality that is commonly applied to reduce the pain, swelling and discomfort associated with local joint or soft-tissue inflammation.

Similarly, massage is well known for its soothing effects on injured or sensitive areas of the body. The beneficial effects of a massage may be combined with cryotherapy and/or heat therapy to provide superior treatment relative to either cryotherapy or massage alone.

### BRIEF SUMMARY

In one aspect of this disclosure, a device for applying compressive hot or cold therapy to a patient's body is disclosed. The device includes a handle and a frame connected to and extending from the handle. The frame includes two spaced apart support arms, the support arms each having an opening such that the openings in the support arms are collinear with one another. A roller having a central axis and a reservoir within its interior for retaining a thermal medium, the roller having a non-uniform cross-section along its length defining a thermally conductive external surface for transferring thermal energy from the thermal medium to the patient's body. At least one elongated member is operatively connected to the roller and extends longitudinally along the central axis beyond the periphery of the roller. The elongated member is received within the collinear openings of the spaced apart support arms to releasably and rotatably connect the roller to the frame.

In another aspect of this disclosure, a method for applying hot or cold message therapy to a patient's body is disclosed. The method comprises providing a thermal medium within a reservoir within a roller having a non-uniform cross-section along its length that defines a thermally conductive external surface for transferring thermal energy from the thermal medium to the patient's body. The roller is heated or cooled to raise or lower the temperature of the thermal medium. The roller is releasably and rotatably connected to two spaced apart support arms of a frame by inserting each end of an elongated member operatively connected and extending along a central axis from the roller through a corresponding opening in the support arms. The roller is rolled over the patient's body by grasping a handle attached to the frame to transfer thermal energy from the thermal medium through the non-uniform, thermally conductive external surface of the roller to the patient's body.

The foregoing has outlined rather generally the features and technical advantages of one or more embodiments of this disclosure in order that the following detailed description may be better understood. Additional features and advantages of this disclosure will be described hereinafter, which may form the subject of the claims of this application.

### BRIEF DESCRIPTION OF THE DRAWINGS

This disclosure is further described in the detailed description that follows, with reference to the drawings, in which:

FIG. 1 is a front plan view of a preferred hot and cold therapy device;

FIG. 2 is a side elevation view of the hot and cold therapy device of FIG. 1;

FIG. 3 is front top plan view of an illustrative flexible belt for use with the hot and cold therapy device of FIG. 1;

FIG. 4 is a front plan view of the hot and cold therapy device roller of FIG. 1 assembled to the flexible belt of FIG. 3;

FIG. 5 is a side elevation view of the assembled hot and cold therapy device roller and flexible belt of FIG. 4;

FIG. 6 is a front cross-sectional view of the hot and cold therapy device taken across line A-A of FIG. 2;

FIG. 7 is a side elevation view of another embodiment of the hot and cold therapy device;

FIG. 8 is a front elevation view of another embodiment of the hot and cold therapy device; and

FIG. 9 is a side elevation view of the hot and cold therapy device of FIG. 8.

### DETAILED DESCRIPTION

This application discloses a hot and cold therapy device 100 that may be used to apply a hot or cold massage therapy to an injured area of the body of a patient. The hot and cold therapy device 100 includes a roller that may be filled with a heated or chilled thermal medium (e.g., liquid) to enable application of a hot or cold message therapy.

Additionally, the hot and cold therapy device 100 may be disassembled, which may be advantageous for a number of reasons. First, it allows a user to heat or chill the thermal medium within the roller without heating or chilling the entire device. Second, it may allow a user to utilize a number of different rollers having various shapes, textures and other characteristics beneficial to a particular application, such as conforming to the shape of the portion of the body to be treated. Switching between rollers may be performed quickly and easily. Third, it allows the roller to be releasably connected to a flexible strap, so that the roller may be held in place against the injured portion of the body. This may allow hands free application of continuous, static hot or cold pressure to the injured area of the body.

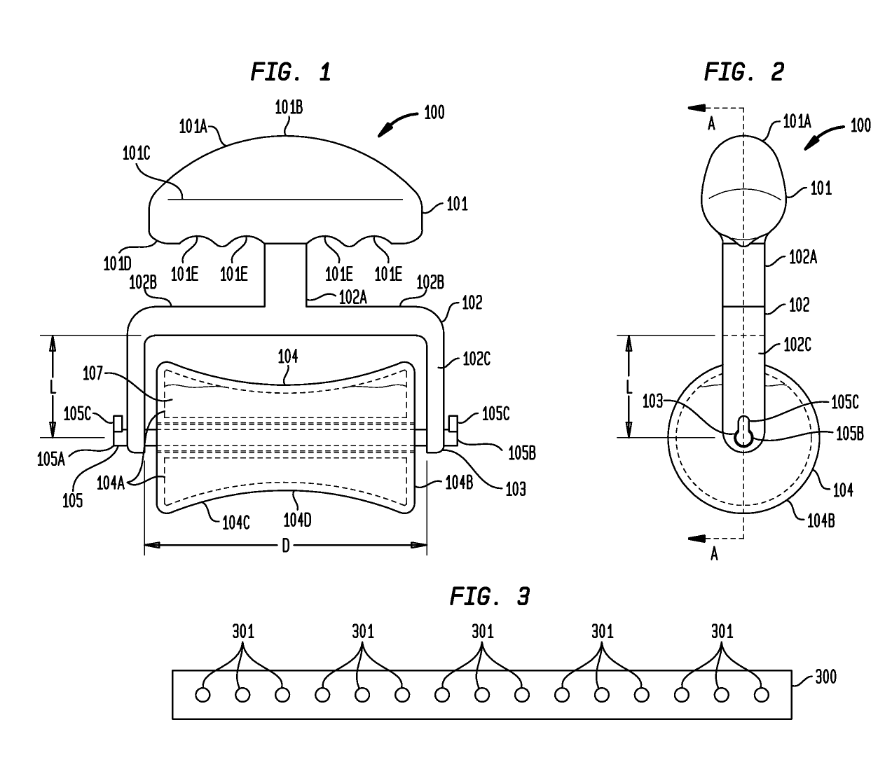

FIGS. 1, 2 and 6 illustrate a preferred, fully assembled hot and cold therapy device 100. The hot and cold therapy device 100 preferably includes a handle 101, a frame 102 and a roller 104. The handle 101 is preferably an ergonomically shaped handle designed for fitting comfortably in a user's hand. Handle 101 is preferably formed of a durable, lightweight, but comfortable to hold material, such as (but not limited to) plastic, rubber, metal, wood, or plastic coated with a rubber-like material. In the preferred embodiment, the hot and cold therapy device 100 is preferably held in the user's palm facing down against the affected area of the patient's body. Handle 101 is preferably ergonomically shaped to comfortably support this kind of “palm down” grip by the user. Handle 101 may, for example, have a semi-circular rounded top curvature 101A. The top curvature 101A preferably starts from a midpoint 101B and slopes gently down to a peripheral edge 101C. A bottom segment 101D of handle 101 may include a plurality of indentations 101E that conform to the user's fingers when the user grips the hot and cold therapy device 100 with a palm down grip. Alternatively, the ergonomic handle 101 may be sufficiently tall and/or wide so that the user's hand and fingers do not contact the bottom surface 101D, in which case, the bottom surface 101D may be flat.

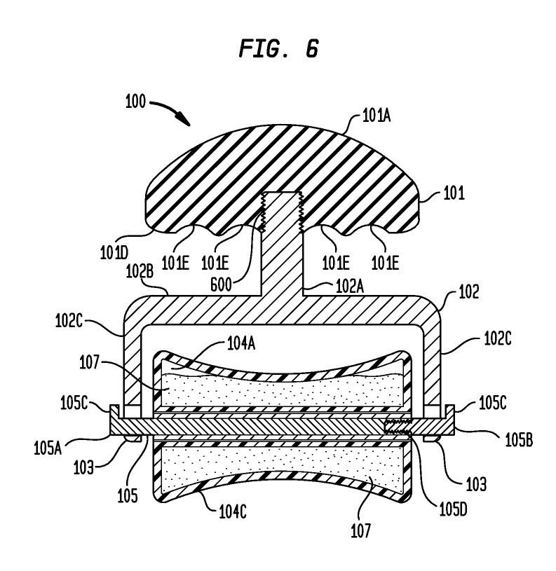

Ergonomic handle 101 and frame 102 may be connected or otherwise assembled together in a conventional manner. The handle 101 and frame 102 may be manufactured as a single component using, for example, conventional injection molding techniques. Alternatively, as best illustrated in FIG. 6, the handle 101 and frame 102 may be assembled as separate components in which, for example, frame 102 may extend through bottom surface 101D and into the body of the ergonomic handle 101. Mating helical threads 600 may be provided on both the frame 102 and handle 101 to releasably connect both components to one another via rotation of one component relative to the other. Alternatively, glue, epoxy, soldering, welding, or one or more mechanical fasteners may be utilized to releasably or permanently connect the handle 101 to the frame 102.

Frame 102 preferably defines the main body of the cold therapy device 100. Frame 102 may be made from any suitable material that is strong enough to withstand the forces typically exerted on the cold therapy device 100 without suffering structural failure. For example, the frame 102 may be made from plastic, metal or wood. Frame 102 may have any desired cross-section or desired combination of cross sections. As depicted in the drawings, frame 102 may have a compact, predominantly square or rectangular cross section. The edges are preferably rounded or smoothed to eliminate sharp edges that may injure a user or patient and to present an aesthetically pleasing finish for the user. Alternatively, circular, ovular, L-shaped, C-shaped or even irregular cross-sections may be utilized as desired.

Frame 102 preferably includes a neck 102A, at least one lateral member 102B and at least one supporting arm 102C projecting or otherwise extending from the terminal end of the lateral member 102B. In one preferred embodiment, the frame 102 includes a pair of lateral members 102B extending laterally from the neck 102A, and a pair of spaced apart supporting arms 102C projecting or otherwise extending from the terminal end of the lateral members 102B. Neck 102A is preferably releasably or permanently connected to ergonomic handle 101. As described above, handle 101 and frame 102 may be integrally formed as a single unit or as separate components in which neck 102A may extend through the lower surface 101D into the body of ergonomic handle 101 and connect to the handle utilizing, for example, mating male and female helical threads 600 within the handle 101 and on the terminal portion of the neck 102A, glue, epoxy or some other adhesive, soldering, welding, or one or more mechanical fasteners.

Neck 102A preferably projects perpendicularly from the lower surface 101D of ergonomic handle 101 and branches laterally into at least one lateral member 102B. In one preferred embodiment, a pair of lateral members 102B extend in opposing directions from neck 102A in a generally perpendicular direction to the neck, thereby forming a single horizontal structure that follows a generally parallel path relative to the plane of lower handle surface 101D.

Each lateral member 102B preferably transitions into a support arm 102C, which is preferably the portion of frame 102 that is releasably connected to roller 104. Each support arm 102C preferably extends in a generally perpendicular direction from lateral member 102B and preferably includes an opening 103 for releasably and rotatably connecting the roller 104 to the frame 102 (described further below). The support arms 102C should be spaced apart by a distance D that is sufficient to accommodate the width of the roller 104 and permit the roller to rotate freely relative to the frame 102 without interference from lateral members 102B. Similarly, the distance L between the underside of the lateral members 102B and the central axis of the opening 103 in the support arms 102C should be sufficient to accommodate the maximum radius of the roller 104 and permit the roller to rotate freely when mounted in the frame 102. It is understood that support arms 102C may extend from lateral members 102B at any desired angle, but the distances D and L should remain sufficient to allow roller 104 to rotate freely without interference when mounted within the frame 102.

Roller 104 is used to apply hot or cold massage therapy to the injured area of the patient's body. Roller 104 is preferably made from a thermally conductive material, such as (but not limited to) metal, plastic, rubber or other pliable (but sufficiently firm) materials. As best illustrated in FIGS. 1 and 6, roller 104 preferably includes a hollow chamber or reservoir 104A for receiving and storing a heatable, chillable and/or freezable heat transferable medium 107. The heat transferable medium may be a non-toxic liquid, fluid, gel, gas, refrigerant or other substance that can be heated and/or cooled to a desired temperature within the roller 104.

The heat transferable medium 107 may be inserted into and sealed within the hollow chamber 104A during manufacture of roller 104. Alternatively, one or more sealable openings (not shown) may be provided in the roller 104, preferably on a surface of roller 104 that does not contact the patient, to allow addition and/or removal of the heat transferable medium 107.

Any suitable heat transferable material or substance may be utilized as the heat transferable medium 107. For example, hollow chamber 104A may be filled with water. The water may then be refrigerated or frozen, thereby providing a reservoir containing a low temperature heat transferable material within roller 104 for a cold therapy massage. Alternatively, more exotic substances may be used. For example, a refrigerant gel may be utilized in place of water as the heat transferable medium 107 within the hollow chamber 104A of roller 104. Refrigerant gels are common materials typically utilized in ice packs. Refrigerant gels may include water and sodium carboxymethyl cellulose, which is a nontoxic, noncarcinogenic mixture that provides temperatures colder than those attainable by water ice, allowing it to remain cold or frozen longer in room temperature environments.

Other heat transferable substances may be utilized as heat transferable medium 107 to produce beneficial effects. For example, a mixture of water and ammonium nitrate may be utilized as heat transferable medium 107. Such mixtures are commonly utilized in “cold packs,” and may be utilized here as well. When water and ammonium nitrate are combined, the resulting mixture generates an endothermic reaction, greatly lowering the temperature of the solution (and thus, the roller 104), which may be beneficial for applying a cold massage therapy with the roller 104.

Alternatively, a solution of sodium acetate (dissolved into its water of crystallization) may be utilized as heat transferable medium 107. Such solutions are commonly utilized in “heating pads,” “hand warmers” and other portable heat emitting devices. A mechanism within roller 104 may allow a nucleation center to be formed within the solution, causing the solution to begin crystallizing. The crystallization process is exothermic, and may thereby emit a large amount of heat, which may be beneficial for applying a heated massage therapy with the roller 104. The sodium acetate solution may thereafter be reused by boiling the solution within the roller 104 to dissolve the sodium acetate trihydrate crystals.

Roller 104 may take a generally cylindrical (but not necessarily uniform) form, with circular top and bottom sections 104B and a cylindrical body 104C. The circular sections 104B may each present a generally flat surface. Cylindrical body 104C preferably includes a concave slope that begins at or near a circumferential edge of a section 104B, which may then gradually decrease down toward a midpoint 104D (or another location) of roller 104. Therefore, in this example, cylindrical body 104C has the smallest cross-sectional diameter relative to the cross-sectional diameters along its entire length (up to a maximum diameter possessed by circular sections 104B) at or near midpoint 104D. This may present a more ergonomic shape to the roller 104, enabling superior application of pressure and heat or cold therapy to the affected region of the patient's body.

Other roller shapes may be utilized as desired to conform to the particular area of the patient's body that is being treated. For example, a roller 104 with a less concave (or even flat) cylindrical body 104C may be utilized for massaging flatter portions of the body, such as the patient's abdominal or back bones or muscles. Alternatively, roller 104 may be formed with a raised or convex cylindrical body 104C, cresting at or near its midpoint (or another location) between the two circular sections 104B. Such a roller 104 may be used, for example, to target a very specific region of the patient's body. Any advantageous shape may be utilized for roller 104 as desired, including combinations of concave and convex configurations for the cylindrical body 104C. Additionally, small shapes, textures or other surface modifications may be added to the cylindrical body 104C to affect a different feeling during the massage or improve the hot or cold therapy.

Referring to FIGS. 1 and 6, an axle or shaft 105 may be utilized to provide an axis for the rotation of roller 104 relative to the frame 102. The roller 104 may include a longitudinally extending opening or bore and optionally a longitudinally extending sleeve or journal 106 may be press fit within the longitudinally extending opening of the roller. An axle 105 may be inserted into the longitudinally extending opening (or into the sleeve or journal 106 if provided) and extend longitudinally through the roller 104 into respective longitudinally aligned openings 103 formed in the support arms 102C of frame 102. Each end of the axle 105 preferably extends beyond the longitudinally aligned openings 103 of frame 102 and preferably includes one or more protrusions 105C, which serve both as a stop to prevent the axle 105 from sliding out of the frame 102 and as a means for releasably connecting the roller 104 to a flexible belt or band 300 if the roller 104 is to be held statically in place against the injured portion of the body (as will be described further below with respect to FIGS. 3-5). The roller 104 may also be manufactured such that there is no central axis, but instead the material used to make the roller is formed in such a manner that the side connectors (105A and B, C) project directly and contiguously from the sides of the roller.

To facilitate convenient and easy attachment/detachment of the roller 104 to the frame 102, at least one end of the axle 105 may include a threaded opening 105D for releasably engaging to mating threads on a threaded fastener or coupling 105B. While the fastener 105B is illustrated in FIG. 6 as having male threads and the axle 500 as having a corresponding female threaded opening 105D, it is understood that the axle could alternatively include the male threads and the fastener could include a corresponding female threaded opening. The threaded fastener 105B preferably has body portion with a diameter that is approximately the same size as the diameter of the axle 105 so that the threaded fastener may be inserted through an opening 103 in the support arm 102C of frame 102. The threaded fastener 105B preferably includes the protrusion 104F at its terminal end in the same manner as described above.

Therefore, during assembly of the hot and cold therapy device 100, axle 105 may be inserted through an opening 103 within one of the support arms 102C and then through the longitudinally extending opening (or sleeve or journal 106 if provided) of roller 104 positioned between the support arms 102C of frame 102. The threaded fastener 105B may then be inserted through the co-aligned opening 103 of the opposing support arm 102C and threaded into the threaded opening 105D at the end of the axle 105, thereby releasably and rotatably connecting roller 104 to frame 102. The roller 104 may be removed from the frame 102 in a similar manner by unscrewing the threaded fastener 105B from the end of the axle 105, after which the axle may be removed from the longitudinally extending opening (or sleeve or journal 106 if provided) of roller 104 and the opening 103 within the support arm 102C through which it passes.

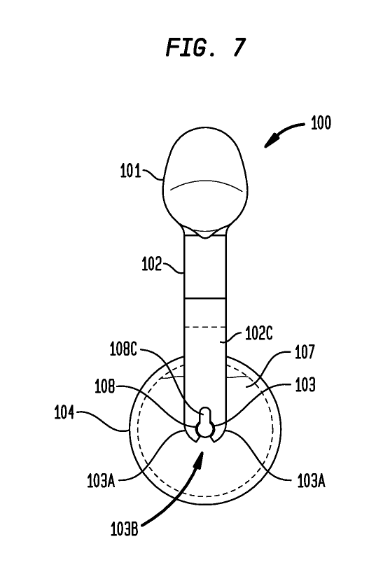

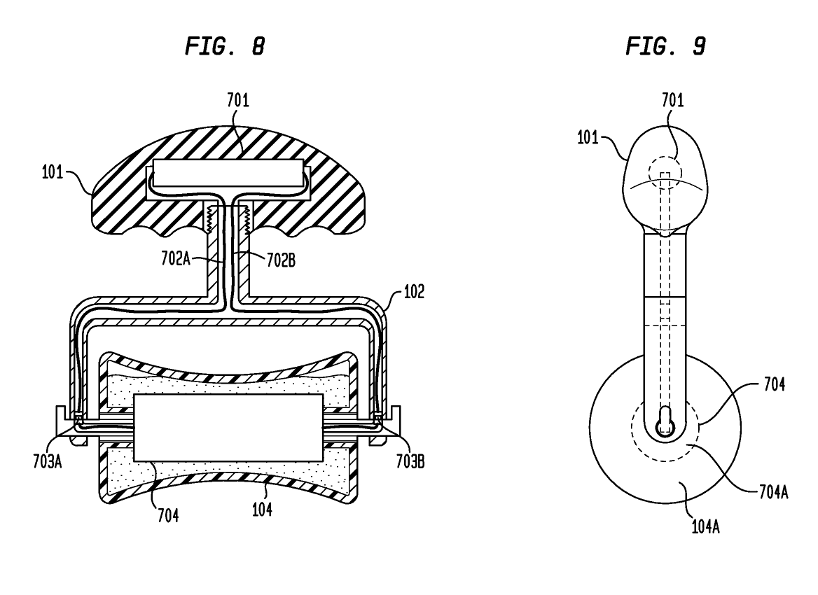

In an alternative embodiment illustrated in FIG. 7, roller 104 may include a pin or cylindrical member 108 extending longitudinally along a central axis from each end of the roller 104 in lieu of the axle 105. A pair of pliable molded jaws 103A located in each supporting arm 102C of frame 102 partially enclose and define the opening 103 in the supporting arms 102C. The pliable molded jaws 103A are preferably separated by a small gap 103B through which the pin or cylindrical member 108 extending from the roller 104 may be forcibly introduced so that the pair of molded jaws 103A flex a sufficient amount to allow the pin or cylindrical member 108 to pass through the gap 103B into the opening 103 partially surrounded by the jaws. Once the pin or cylindrical member 108 is inserted within the opening 103 surrounded by the jaws 103A, the jaws snap back or elastically return to their original configuration to retain the pin or cylindrical member 108 within the opening 103 partially surrounded by the jaws to thereby releasably and rotatably connect the roller 104 to the support arms 102C. Roller 104 may similarly be removed from the frame 102 by applying a force on the pin or cylindrical member 108 sufficient to cause the pair of pliable jaws 103A to flex away from one another and permit the pin or cylindrical member 108 to pass through the gap 103B between the jaws. The pliable jaws 103A may be formed in the support arms 102C in a conventional manner, such as, for example, during a molding process for manufacturing frame 102. It is understood that other mechanisms for releasably and rotatably connecting the roller 104 to the frame 102 may be utilized as well. The pin or cylindrical members 108 extending longitudinally from the roller 104 may also include one or more protrusions 108C (similar to protrusions 105C described above) to releasably connect the roller 104 to a flexible strap 300 when the roller 104 is to be held statically in place against the injured portion of the body (as will be described below with respect to FIGS. 3-5).

FIG. 3 illustrates the flexible strap or tape 300 that may be utilized in conjunction with the roller 104 to apply static hot or cold pressure against an area of the patient's body. Flexible strap or tape 300 preferably includes a plurality of openings 301 centrally aligned along the strap's longitudinal axis. The flexible strap or tape 300 may be a flexible strap, belt, band, bandage or tape made of any suitable material, such as (but not limited to) cloth athletic tape, a woven elastic bandage or fabric, or the like.

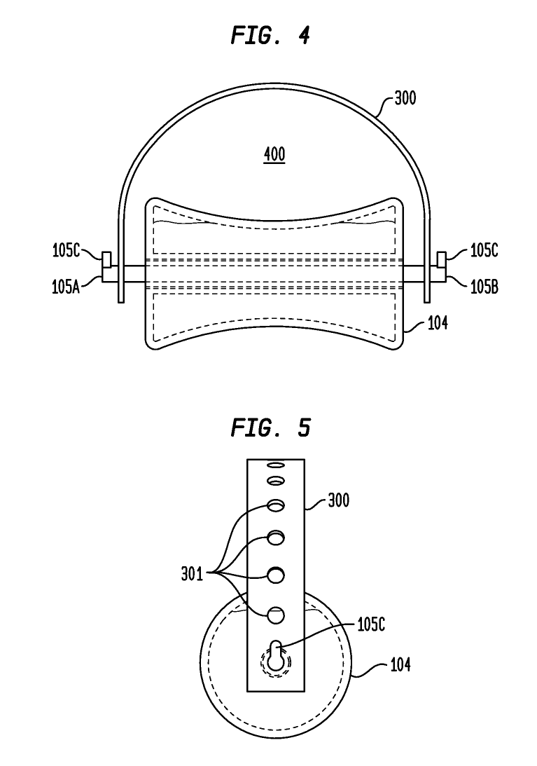

FIGS. 4 and 5 illustrate the flexible strap 300 releasably connected to the roller 104 to maintain the roller in a static position against an area of a patient's body to be treated. The flexible strap 300 may be releasably connected to the roller 104 by inserting the end 105A of the axle 105 and the threaded fastener 105B into respective openings 301 within the strap 300. The protrusions 105C extending from the ends 105A of the axle 105 and the threaded fastener 105B extend beyond the circumference of openings 301 to thereby prevent the strap 300 from unintentionally disconnecting from the roller 104. When assembled in this manner, the flexible strap 300 and roller 104 define an open region 400. The roller and strap assembly may therefore be wrapped around a body part, such as an arm or leg, in which the body part is positioned within the loop 400. The roller and strap assembly may then be tensioned against the body part by selecting the appropriate opening 301 such that sufficient tension exists to statically maintain the roller 400 in place against the body part to be treated. In this manner, pressure and hot or cold therapy may be statically applied to the designated area.

Prior to use, the heat transfer medium 107 within the roller 400 may be heated or cooled to a desired temperature by placing the thermally conductive roller in a suitable heating or cooling device, such as (but not limited to) an oven, microwave, refrigerator or freezer. Once the roller 400 (and the heat transfer medium 107) have achieved the desired temperature, the roller may either be releasably and rotatably connected to the frame 102 so that a user may apply the hot or cold therapy to the injured area of the patient's body by massaging the injured area with the thermally conductive roller. Alternatively, the thermally conductive roller 400 may be positioned against the injured area of the patient's body and the flexible strap 300 may be wrapped about the injured area and releasably attached to the roller to apply static hot or cold therapy to the injured area of the patient's body.

FIGS. 7 and 8 illustrate another embodiment of the hot and cold therapy device 700. The cold therapy device 700 may be equipped with an electrically powered mechanism 704 for providing additional effects for a massage therapy. For example, the electrically powered mechanism 704 may include a vibratory motor that vibrates within the roller 104, enabling the addition of vibration to a massage therapy. The electrically powered mechanism 704 may optionally include an electrically operated heating element for heating the heat transferable medium 107 within the roller 104 and/or the roller itself, allowing one to potentially alter or control the temperature of a cold massage, or even apply (or transition to) a hot massage.

Ergonomic handle 101 preferably houses a power source 701 for supplying power to the electrically powered mechanism 704. Standard battery solutions may be utilized for power source 701. For example, power source 701 may be one or more standard replaceable batteries or rechargeable batteries. Alternatively, power source 701 may be permanently installed in ergonomic handle 101. Charging of the power source 701 (e.g., rechargeable battery(s)) may be achieved via an externally attachable charging cable. In yet another embodiment, a motion-charged battery may be utilized, allowing motion from an applied massage to at least partially recharge the battery.

Power source 701 is preferably electrically connected to the electrical mechanism 704 via electrical circuit 702a and 702b. Electrical circuit 702a and 702b may be housed within frame 102. Electrical contacts or connectors 703a and 703b may complete electrical continuity with the electrically powered mechanism 704, which is preferably housed within the body of roller 104. In this manner, the roller 104 may be removed from the frame 102. The electrical contacts or connectors 703a and 703b may be designed to provide continuous continuity even as the roller 104 rotates relative to frame 102. A separate chamber may be included in the body of roller 104 for the electrically powered mechanism 704, thereby ensuring proper function of the mechanism 704 by segregating any fluids from the mechanism 704 and electrical contacts 703a and 703b.

Having described and illustrated the principles of this application by reference to one or more preferred embodiments, it should be apparent that the preferred embodiment(s) may be modified in arrangement and detail without departing from the principles disclosed herein and that it is intended that the application be construed as including all such modifications and variations insofar as they come within the spirit and scope of the subject matter disclosed.

What is claimed is:

1. A device for applying compressive hot or cold therapy to a patient's body, comprising:
a handle;
a frame connected to and extending from the handle, the frame including two spaced apart support arms, the support arms each having an opening such that the openings in the support arms are collinear with one another;
a roller having a central axis and a reservoir within its interior for retaining a thermal medium, the roller having a non-uniform cross-section along its length defining a thermally conductive external surface for transferring thermal energy from the thermal medium to the patient's body; and
at least one elongated member operatively connected to the roller and extending longitudinally along the central axis beyond the periphery of the roller, the elongated member being received within the collinear openings of the spaced apart support arms to releasably and rotatably connect the roller to the frame.

2. The device of claim 1, wherein the roller has a generally concave cross-section along its length.

3. The device of claim 1, wherein the roller has a generally convex cross-section along its length.

4. The device of claim 1, wherein the elongated member comprises an axle that extends longitudinally along the central axis through the roller.

5. The device of claim 4, further comprising a threaded coupling that releasably engages corresponding threads on at least one end of the axle to releasably connect the roller to the frame.

6. The device of claim 1, wherein the elongated member includes a pin that extends longitudinally from a lateral edge of the roller along the central axis.

7. The device of claim 6, wherein the elongated member includes a second pin that extends longitudinally from an opposing lateral edge of the roller along the central axis.

8. The device of claim 1, wherein the opening in each support arm is defined and partially surrounded by two flexible opposing jaws that are separated by a gap, the opposing jaws being movable between a normally closed position in which the gap is smaller than the elongated member to retain the elongated member within the opening and an open position in which the gap is larger than the elongated member to permit the elongated member to be inserted into or removed from the opening.

9. The device of claim 1, further comprising a flexible elastic strap having a plurality of openings along its length for receiving an end of the elongated member when the roller is removed from the frame, wherein the roller may be statically maintained in position against the patient's body by wrapping the strap about the patient's body and inserting each end of the elongated member into a respective opening in the flexible elastic strap.

10. The device of claim 9, wherein each end of the elongated member includes a protrusion extending transversely therefrom to prevent inadvertent removal of the elongated member from the opening in the flexible elastic strap.

11. The device of claim 1, wherein the frame further comprises a neck that connects to the handle at one end and terminates in a pair of lateral members extending laterally from the neck, each lateral member having a terminal end from which the supporting arm extends.

12. The device of claim 1, wherein the handle includes a semi-circular rounded top curvature and a bottom segment having a plurality of indentations that conform to a user's fingers when the user grips the handle.

13. The device of claim 1, wherein the roller is adapted to be heated to elevate the temperature of the thermal medium prior to releasably connecting the roller to the frame.

14. The device of claim 1, wherein the roller is adapted to be cooled to lower the temperature of the thermal medium prior to releasably connecting the roller to the frame.

15. The device of claim 1, wherein the thermal medium comprises a refrigerant gel.

16. The device of claim 1, wherein the thermal medium comprises a mixture of water and ammonium nitrate.

17. The device of claim 1, wherein the thermal medium comprises sodium acetate.

18. The device of claim 1, further comprising a vibration motor positioned within the roller.

19. The device of claim 18, further comprising a power source electrically connected to the vibration motor for supplying power to the vibration motor.

20. The device of claim 19, wherein the power source comprises at least one battery positioned within the handle.

21. The device of claim 1, further comprising a heating element positioned within the roller.

22. The device of claim 21, further comprising a power source electrically connected to the heating element for supplying power to the heating element.

23. The device of claim 22, wherein the power source comprises at least one battery positioned within the handle.

24. A method for applying hot or cold message therapy to a patient's body, comprising:
providing a thermal medium within a reservoir within a roller having a non-uniform cross-section along its length that defines a thermally conductive external surface for transferring thermal energy from the thermal medium to the patient's body;
heating or cooling the roller to raise or lower the temperature of the thermal medium;
releasably and rotatably connecting the roller to two spaced apart support arms of a frame by inserting each end of an elongated member operatively connected and extending along a central axis from the roller through a corresponding opening in the support arms; and
rolling the roller over the patient's body by grasping a handle attached to the frame to transfer thermal energy from the thermal medium through the non-uniform, thermally conductive external surface of the roller to the patient's body.

25. The method of claim 24, further comprising:
removing the roller from the frame;
positioning the roller against the patient's body;
wrapping a flexible elastic strap around the patient's body; and
inserting each end of the elongated member into a corresponding opening in the flexible elastic strap to hold the roller in a static position against the patient's body.

26. The method of claim 24, further comprising threading a fastener into at least one end of the elongated member to releasably retain the elongated body within the corresponding opening in the support arm.

27. The method of claim 24, further comprising:
positioning each end of the elongated member between two flexible opposing jaws formed on each support arm, the flexible opposing jaws being separated by a gap and partially surround the opening on each support arm;
applying pressure on each end of the elongated member to cause the opposing jaws move from a normally closed position in which the gap is smaller than the elongated member to an open position in which the gap is larger than the elongated member to permit the elongated member to be inserted into the opening.

28. The method of claim 24, further comprising energizing a vibration motor positioned within the roller to cause the roller to vibrate.

29. The method of claim 24, further comprising energizing a heating element positioned within the roller to heat the thermal medium.
